(19) United States
(12) Patent Application Publication
(10) Pub. No.: US 2006/0286076 A1
(43) Pub. Date: Dec. 21, 2006

(54) Device and method for applying active substances to the surface of a wound

(76) Inventor: Wilhelm Fleischmann, Bietigheim-Bissignen (DE)

Correspondence Address: AKERMAN SENTERFITT, P.O. BOX 3188, WEST PALM BEACH, FL, 33402-3188, US

(21) Appl. No.: 10/571,222

(22) PCT Filed: Aug. 18, 2004
(86) PCT No.: PCT/EP04/09243

§ 371 Date: Jul. 21, 2006

(30) Foreign Application Priority Data
Sep. 10, 2003 (DE) 103-42-071.1

(51) Int. Cl. A61K 35/76; A61M 31/00

(52) U.S. Cl. 424/93.6; 604/500

(57) ABSTRACT

The invention relates to the application of active substances to the surface of a wound. An insert made of porous material is applied to the surface of the wound, and a sealing overlay is used to cover the surface of the wound and the inlay. The liquid active substance is fed in a temporally controlled manner into the insert and then is suctioned. The liquid active substance contains bacteriophages in order to improve healing of the wound.

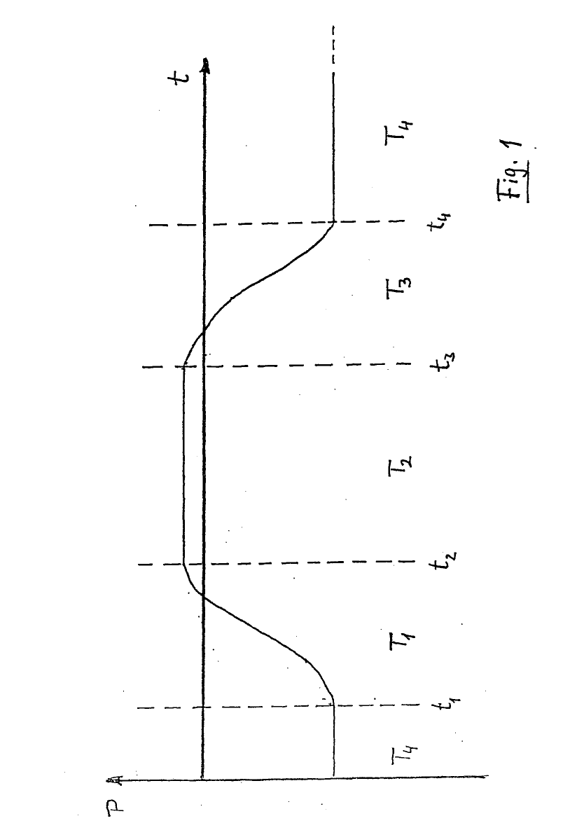

### CROSS REFERENCE TO RELATED APPLICATION

This application is a national stage of PCT/EP2004/009243 filed Aug. 18, 2004 and based upon DE 103 42 071.1 filed Sep. 10, 2003 under the International Convention.

### BACKGROUND OF THE INVENTION

1. Field of the invention

The invention concerns a device and a method for applying active substances to the surface of a wound.

2. Description of Related Art

A device of this type for application of active substances to a wound surface is known from DE19722075C1 (U.S. Pat. No. 6,398,767). With this known instillation system substances can be applied to the outer surface of a wound in order to be active on the wound surface over a controllable span of time. After this active time interval the active substance is suctioned off, and in certain cases a partial vacuum can be maintained for a subsequent time interval.

### SUMMARY OF THE INVENTION

The invention is concerned with the task of providing a new type of wound treatment.

Advantageous embodiments of the invention are set forth in the dependent claims.

The invention takes advantage of the activity of bacteriophages on bacterial infections.

Bacteriophages, also known also known as phages, are viruses, of which the host cells are bacteria. They can penetrate into the bacteria and multiply therein. In the case of lysogenic bacteria phages the bacteria can survive, while with lytic bacteria phages the bacteria would be destroyed. Lytic bacteria phages are thus used for treatment of bacterial infections. Therein it is necessary to employ as the bacteriophages viruses with the highest possible virulence against the target bacteria. Particularly suited for the treatment of an infection with gram negative pathogens seem to be the bacteriophages of the T-even group according to the Ackermann type classification. In comparison to treatment with broad spectrum antibiotics, the treatment with bacteriophages has the advantage that the bacteriophages, due to their pathogen specificity, have hardly any side effects. The bacteriophages can also kill germs that are resistant to antibiotics. As the number of multi-resistant infectious pathogens, which now no longer respond to any antibiotic, increases, the bacteriophage therapy assumes steadily increasing importance.

Bacteriophages have a series of characteristic features. They are highly specific, that is, they selectively infect only certain bacteria. They require an alkali environment and are destroyed in an acidic environment. They require a relatively narrowly defined range of environmental temperature, for example approximately 37° C. They replicate exponentially, until their nutrient reserve is depleted, that is, until the target bacteria are eliminated. The bacteriophages can remain dormant in lifeless rest phases (virions) typical for viruses, until a renewed contact with a specific receptor of a bacterial cell sets their reproduction into gear. The bacteriophages can transport resistance genes and toxin genes and increase their effect by the action of bacterial toxins, which can lead to the dangerous Herxheimer reaction. In systemic applications of the phages there is only a short bioavailability, since a rapid decomposition occurs by the reticulo-endothelial system, in particular by the spleen.

### DETAILED DESCRIPTION OF THE INVENTION

The invention concerns a new way in which the characteristics of the bacteriophages can be employed for wound treatment, while preventing the harmful characteristics from causing hazardous consequences.

The course of wound treatment and the manner of operation of the device are explained in greater detail on the basis of the figure, wherein the single figure represents in a diagram the pressure T in a wound as a function of time in the inventive process. The abscissa therein represents the atmospheric pressure.

An inlay of a porous material, for example an elastic compressible open-pore sponge material, is introduced into the wound to be treated. The wound surface and the inlay are covered over with a sealing overlay, for example a sheet or a foil, which is secured sealingly to the wound surface around the edges of the wound. In the inlay there is supply and a drainage line. The supply line and drainage line are provided with controllable shut-off valves. A supply of liquid active material is supplied via the supply line, during which a partial vacuum source can be connected to the drainage line, in order to draw the fluid out of the wound and, in particular, the inlay.

In the diagram shown in the figure, at time t₁ the shut-off valve of the supply line and the drainage line are closed. In the wound there is some amount of partial vacuum, which could be for example 10-80 kPa. On the basis of this partial vacuum the sealing foil is pressed against the wound surface, at which time the elastic porous inlay is compressed. At time T₁ the shut-off valve at the supply line is controlled to open, so that the liquid active agent with the bacteriophages can flow via the supply line into the inlay and therewith the wound. During the inflow time interval T₁ the inlay draws itself full of the liquid active agent, at which time the inlay expands due to spring-elastic return force. At time t₂ the inlay is drawn full of the liquid active agent, at which time a certain amount of positive pressure exists beneath the foil, as determined for example by the elevation of the supply container relative to the wound. In certain cases it would also be possible to switch on a pressure controlled pump to the supply line.

As soon as the inlay has drawn itself full of the liquid active agent, at time t₂ the shut-off of the supply line is closed. For the active period time interval T₂ (instillation or hold phase) the shut-offs of the supply line and drainage line remain closed, so that the active agent contained in the inlay can act upon the surface of the wound. The duration of this exposure or active phase can be controlled. It is possible in association therewith to also provide one or more sensors in the wound or, as the case may be, the device, which measures the concentration of the bacteriophages and/or the pH value and/or the temperature. After expiration of the exposure phase at time t₃ the shut-off of the device is opened, so that as a result of the existing partial pressure the liquid active agent is suctioned out of the inlay and the wound in interval T₃. If at time t₄ the original partial vacuum is again established, then the liquid active agent is completely removed out of the wound and the inlay and the partial vacuum is now maintained again over the vacuum time interval T₄. During this time the shut-off valve of the drainage line can remain open, so that the partial vacuum can be continuously maintained.

During the active time interval T₁/T₂, that is, the installation/hold phase, the bacteriophages flow, driven by pressure, into the liquid spaces as well as through tissue septum and lymph nodes of the infected tissue in which the bacteria also multiply. The bacteria are lysed by the bacteriophages and release their dangerous toxins. During the subsequent vacuum interval T₃/T₄ there occurs a partial pressure reversal, and therewith also flow reversal, and disrupted bacteria with their toxins are suctioned out of the tissue before they can damage the organism. Thereby there is prevented for example a toxic shock due to a Herxheimer-reaction. The time interval of pressure and vacuum phases T₂ to T₄ are determined in accordance with clinical monitoring and scientific data regarding toxin release. A strong toxin release requires short activity intervals T₂ and long vacuum phases T₄. Alternatively or supplementally the phage concentration can be varied in the installation fluid, that is, in this case it can be reduced.

The removal of phages out of the infected tissue during the vacuum phase T₃/T₄ prevents or reduces also their crossing over into the blood and lymph circulation. Immuno reactions of organism, which lead to the recognition of and destruction of virus, are thereby delayed and the local bio availability of the phages is elevated. In the same manner the phage-containing installation liquid has a protective function. It drives away or reduces, at least during the active interval T₂, the immunologically active tissue fluids which cause an inactivation of the phages.

The inventive installation can also be employed for systemic phage therapy. During the active phase T₂ the phage concentrate is introduced via the wound surface into the body tissue with the desired pressure therefore, such that systemic phage levels occur. The environment conditions for the phages in the applied installation liquid can be monitored and, in certain cases, be corrected. It is particularly simple to refresh the local phage liquid by short time interval suctioning (T₃/T₄) and subsequently installation (T₁/T₂) of new viral solution. The drop of the phage concentration in the wound or as the case may be the device (phage pool) is corrected with the amount of phages which have transferred into the organism. In order to increase the systemic bioavailability, it is advantageous, among other things, to employ specially bred virus, which are less susceptible to a disruption by the reticulo-endothelial defense system of the organism.

The invention makes possible, besides the described controllable detoxication, the optimal adjustment of phage concentration, pH and temperature. This can have a significant influence on the therapeutic phage activity, since inflammatory reactions of the body tissue lead to an elevation in temperature, which—just as an infection-determined acidic tissue reaction—causes the phages to become inactive.

1-12. (canceled)

13. A device for application of active substances onto a wound surface, the device comprising:
an inlay of a porous material for laying upon the wound surface,
a sealing overlay for covering the wound surface and the inlay, which overlay is sealingly secured to the skin surface,
at least one supply line for a liquid active substance leading into the inlay, which supply line includes a controllable shut-off valve,
at least one drainage line leading from the inlay, which drainage line is connectible to a source of partial vacuum and which drainage line includes a controllable shut-off valve, and
a controller configured to control the shut-off valves such that the shut-off valve of the supply line and the shut-off valve of the drainage line are not simultaneously overlappingly opened and such that an active time interval is established in between the closures of the shut-off valve of the drainage line,
wherein the liquid active substance contains bacteriophages.

14. A device according to claim 13, wherein the bacteriophages are lytic bacteriophages.

15. A device according to claim 13, wherein the inlay is comprised of an elastic compressible porous material.

16. A device according to claim 15, wherein the inlay is comprised of an open-pore foam material.

17. A device according to claim 13, wherein the controller determines, following drainage or suctioning, a partial-vacuum time interval in which a predetermined partial-vacuum is maintained in the inlay.

18. A device according to claim 13, wherein at least one sensor is introduceable under the sealing overlay, which sensor is operatively connected to the controller and measures at least one of the bacteriophage concentration, the pH value, and the temperature.

19. A device according to claim 13, wherein at least one of the bacteriophage concentration, the pH value, and the temperature of the liquid active agent is adjustable.

20. A process for application of active substances to a wound surface, said process comprising:
providing an inlay of a porous material covering in two dimensions upon the wound surface and the inlay
providing a sealing overlay covering over and sealing the inlay around the edges of the wound,
introducing at least one liquid active substance via at least one supply line into the porous inlay
suctioning the liquid active substance out of the inlay via at least one drainage line,
wherein the introduction and the suctioning of the active substance is time-wise controlled to be in separate time intervals (T₁ or as the case may be T₃),
wherein between the introduction and the suctioning an active time interval T₃ is established, and
wherein the liquid active substance contains bacteriophages.

21. A process according to claim 20, wherein the bacteriophages are lytic bacteriophages.

22. A process according to claim 20, wherein subsequent to the suctioning (T₃) of the active substance until the next introduction (T₁) a vacuum time interval (T₄) is established, in which a partial vacuum is maintained under the covering overlay.

23. A process according to claim 20, wherein at least one of the bacteriophage concentration, the pH value and the temperature of the introduced active substance is controlled.

24. A process according to claim 23, wherein at least one of the phage concentration, the pH value and the temperature is measured at the wound surface and used for controlling the introduction of the active substance.
